(19) United States
(12) Patent Application Publication
(10) Pub. No.: US 2006/0134164 A1
(43) Pub. Date: Jun. 22, 2006

(54) Th2-inducing adjuvant treatment for osteolysis

(76) Inventors: Thomas M. DiMauro, Southboro, MA (US); Mohamed Attawia, Canton, MA (US); Chantal Holy, Raynham, MA (US); Hugo Pedrozo, Silver Lake, IN (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/015,926

(22) Filed: Dec. 17, 2004

(51) Int. Cl. A61K 9/14; A61F 2/28

(52) U.S. Cl. 424/422; 424/489; 623/16.11

(57) ABSTRACT

Active immunization and a formulation for treating or preventing osteolysis caused by Wear Particles, comprising: a) a Th2 -inducing adjuvant, and b) Wear Particles.

### BACKGROUND OF THE INVENTION

Minute particles emanating from either ultra high molecular weight polyethylene (UHMWPE) or titanium interfaces or polymethyl methacrylate (PMMA) cement or other wear bearing surfaces cause an inflammatory immune response resulting in osteolysis. Osteolysis is believed to be a primary cause of implant revision in hip and knee implants, and may be a cause for revision of spinal motion discs.

Conventional means of treating osteolysis include: a) providing smoother wear surfaces; b) providing more wear resistant UHMWPE; c) a continuous infusion pump or gene technology as a means of providing IL-10; and d) prosthetic revision surgery.

Some of the osteolysis literature has suggested that the complexing of polyethylene and IgG leads to a Th1-type pro-inflammatory response. It has been suggested in the literature that certain implanted polymers such as silicone may provide an adjuvant-like activity to native macromolecules, which adhere to hydrophobic surfaces and subsequently become immunogenic. Kossovsky, CRC Crit. Rev. Biocompat. 3, 53-85, 1987. In regards to UWMWPE, Wooley, JBJS, 81-a (5) May 1999, 616-623, has suggested that most hip joint prosthesis patients express antibodies that are reactive with the proteins bound to polyethylene and that type I collagen is a major antigenic target in these patients. Wooley reported that type I collagen was often found bound to polyethylene particles, and further suggested that the implantation of the biomaterial, followed by deposition of collagen, may contribute to increased levels of antibodies. Wooley then hypothesizes that immunoglobulin complexed with polyethylene may fix complement and that the complement cascade may in turn attract inflammatory cells to the polyethylene surface. Stuart, J. Exp. Med., 155, January 1982, 1-16, reports that IgG anticollagen antibodies can cause arthritis. Bosetti , Biomaterials, 24, 2003, 1419-26 reports the adsorption of pro-inflammatory IgG upon the surface of UHMWPE.

Accordingly, the literature suggested not only that the binding of collagen to polyethylene but also the binding of IgG to polyethylene may drive a pro-inflammatory response.

Another portion of the osteolysis literature has suggested that the complexation of polyethylene and IgG leads to a Th2-type anti-inflammatory response. For example, Anderson, J. Imunology, 2002, 168:3697-3701, (“Anderson I”) reports that whereas use of macrophages as antigen-presenting cells (APCs) resulted in a strong polarized T cell response predominated by Th1 cytokines, when the antigen was targeted to FC_{χ} receptors on these APCs, the T cell response was reversed and biased toward a Th2-type response. Anderson I concludes that when APCs encounter immune complexes, their cytokine production is modulated to create a cytokine microenvironment which preferentially induces a Th2-like response dominated by IL-4, and that IgG can override innate signals generated by microbial products and drive Th2-like immune responses. Anderson, J. Endotoxin Research 8(6), 2002, 477-481, (“Anderson II”) reports that cells exposed to IgG immune complexes generate large amounts of IL-10, and as a result exert a potent anti-inflammatory effect on the immune response. Anderson II further reports that the ligation of FC_{χ} receptors on activated macrophages by antigen -IgG complexes induced T cells to produce IL-4, which in turn induced B cells to produce IgG1 (a Th2 IgG) in response to that antigen. Anderson II concludes that the mechanism by which IgG can influence immune deviation is by changing the phenotype of the APC, inducing the production of IL-10 instead of IL-12.

Accordingly, the literature suggests that the production of UHMWPE wear debris may drive both Th1 and Th2 responses. The suggestion of a mixed response is consistent with the reporting of Arora, JBMR 64A: 693-697, 2003. Arora examined the specific role of lymphocytes in the Th1 and Th2 subsets in osteolysis and aseptic loosening and found significant numbers of T cells and Th1 and Th2 immune cytokines, and concluded there was a possibility of an immune response at the prosthetic interface.

Since it is likely that the production of UHMWPE wear debris invokes a mixed type immune response involving both Th1 and Th2 cells and both pro- and anti-inflammatory cytokines, the present inventors believe that the presence of a significant Th1 component in the immune response is responsible for the induction of osteolysis.

### SUMMARY OF THE INVENTION

In one embodiment, the present inventors have developed inventions directed towards polarizing the immune response to wear particles (defined below) such as UHMWPE wear debris to a substantially exclusive Th2 response. The present inventors believe that providing a polarized Th2 response will eliminate the Th1 component in the immune response and thereby eliminate osteolysis.

Alum has been used as an adjuvant for many years in vaccinations as a means of provoking Th2 polarization of the immune response:

Mattson, Scand. J. Immunol., 46, 619-24, 1997, studied collagen-induced arthritis development in DA rats after immunization with alum adsorbed to collagen type II. Mattson reported that such immunization treatments suppressed disease development both prophalactically and therapeutically. Mattson reported choosing alum as the adjuvant in order to evoke a Th2 profile, and concluded that such a change occurred because there was a significant increase in IL-4 production and in the IgG1 anti-CII antibody response. Mattson further concluded that it was probable that pretreatment with alum-collagen II primes the immune system to produce Th2 instead of a Th1 response to collagen/FIA immunization, which normally causes arthritis.

Brewer, J. Immunology, 1999, 163: 6448-6454, reports that alum adjuvants can induce IL-4 production and a Th2 response even in the absence of IL-4 signaling in mice deficient in either Il-4a or Stat6.

Comoy, International Immunolouy, 9(4), 523-531, reports immunizing mice with parasitic or bacterial protein antigens in combination with different adjuvants and reported that immunization with either protein antigen in alum induced a strong Th2-associated antibody (IgG1) and cytokine (IL-4) response. Comoy concluded that contrasting cytokine profiles could be induced against the same antigen, depending upon the adjuvant employed.

Cribbs, International Immunology, 15(4), 505-514, immunized wild-type mice with AB antigen in four adjuvants including alum. Cribbs reported that whereas three of the adjuvants provoked a Th1 response, alum provoked a Th2 response. Cribbs concluded that the choice of adjuvant may be critical for the design of a safe and effective immunotherapy for Alzheimer's Disease.

In another embodiment, present invention relates to a preventative method of treating osteolysis by active immunization with Wear Particles and alum adjuvant. In yet another embodiment, at the time the implant is put in place, a small quantity of antigenic Wear Particles are delivered to the implant in an adjuvant capable of evoking a polarized Th2 immunogenic response. Thereafter, the patient becomes immunized against the further creation of the Wear Particles. When Wear Particles are later created by the articulation in much greater quantities, the patient's immune system quickly reacts with a strong Th2 humoral immune response. The humoral immune response is characterized by a large IgG1 antibody production, which provides a means of attacking the Wear Particles produced by the articulation. The humoral response is also characterized by antagonism of Th1 cytokines such as TNF-α and IL-1β and by increased IL-10 production, which is known to antagonize osteoclasts, and so is protective of bone.

Therefore, the present invention not only allows for the clearance of the Wear Particles, it does so in a manner that both eliminates inflammation and protects against osteolysis.

A hallmark of the Th2 immune response is the production of IL-4 and IL-10 from Th2 cells.

It is believed that IL-10 possesses a number of features that make it an attractive therapeutic agent for treating or preventing osteolysis. These include the inhibition of pro-inflammatory cytokine synthesis and the down-regulation of antigen-presenting cell function:

According to Brennan, Rheumatology 1999, 38, 293-7, IL-10 can induce the production of cytokine inhibitors, including the IL-1 receptor antagonist (IL-I1ra) and the release of both soluble TNF receptors p55 and p75 in monocytes. Because of this utility, Brennan chartacterizes IL-10 as a ‘macrophage deactivating factor’.

According to Hart, Immunology, 1995, Apr 84 (4) 536-42, IL-10 and IL-4 have the capacity to downregulate both pro-inflammatory molecules TNF-a and IL-1β. Pollice J. Orthop. Res. 1998 Nov. 16(6) 697-704 discloses that IL-10 inhibits inflammatory cytokine synthesis by monocytes stimulated with titanium particles. Trindade, Biomaterials 22 (2001) 2067-73 discloses that IL-10 inhibits PMMA-induced IL-6 and TNF-a release by human monocytes/macrophages in vitro.

Goodman, JBMR, 65A:43-50, 2003 used a small infusion pump to continuously provide IL-10 to a site contaminated with UHMWPE particles and found that local infusion of immune-modulating cytokines such as IL-10 may prove to be useful in abating particle-induced periprosthetic osteolysis. Carmody, Arthritis & Rheumatism, 46(5) May 2002 pp. 1298-1308 teaches viral IL-10 gene inhibition of inflammation, osteoclastogenesis and bone resorption in response to titanium particles.

Therefore, in accordance with the present invention, there is provided a method of preventing or treating osteolysis from Wear Particles of an orthopedic implant, comprising the steps of:

—administering to a patient a formulation comprising:

a) a Th2-inducing adjuvant, and

b) Wear Particles.

Also in accordance with the present invention, there is provided a formulation for treating or preventing osteolysis caused by Wear Particles, comprising:

a) a Th2-inducing adjuvant, and

b) Wear Particles.

### DETAILED DESCRIPTION OF THE INVENTION

In general, “Wear Particles” includes a) actual wear particles produced from the articulation of two surfaces, and b) particles having a composition and particle size distribution substantially similar to actual wear particles produced from the articulation of two surfaces. For example, “Wear Particles” includes UHMWPE particles produced from a physiologic articulation of a UHMWPE acetabular cup and a prosthetic femoral head, and b) particles having a composition and particle size distribution substantially similar to actual wear particles produced from the physiologic articulation of a UHMWPE acetabular cup and a prosthetic femoral head so as to cause an osteolytic response.

In some embodiments, the Wear Particles are titanium or a titanium alloy. This will allow immunization for Wear Particles emanating from articulation surfaces comprising titanium. In some embodiments, the Wear Particles are cobalt-chrome. This will allow immunization for Wear Particles emanating from articulation surfaces comprising cobalt-chrome. In some embodiments, the Wear Particles are UHMWPE. This will allow immunization for Wear Particles emanating from articulation surfaces comprising UHMWPE, such as those in UHMWPE acetabular cups or tibial components. In some embodiments, the Wear Particles are PMMA. This will allow immunization for particles emanating from cemented surfaces.

In some embodiments wherein the articulating implant has a UHMWPE articulating surface opposing a metal articulating surface, the Wear Particles may be a mixture comprising metal particles and UHMWPE particles. In some embodiments, the mixture comprises about 75 wt % UHMWPE particles and about 25 wt % metal particles.

Generally, the Wear Particles of the present invention are characterized by a D₅₀ particle size of between 0.1 μm and 10 μm, preferably between 0.3 μm and 5 μm, more preferably between 0.5 μm and 3 μm. Preferably, the Wear Particles are provided in a particle size distribution substantially corresponding to the particle size distribution that are produced at the articulation interface of the prosthetic component during wear and then targeted for attack by the immune system. For example, in one embodiment, the antigen is 1-2 μm UHMWPE particles.

Generally, the Wear Particles of the present invention are present in a concentration of about 10⁷ particles/cc to 10¹³ particles/cc.

The amount of Wear Particles in the formulation of the present invention should be sufficient to allow antigen presenting cells to provide the proper signaling to T cells to activate a Th2 immune response, but not so much as to cause an exaggerated immune response. For example, it is believed that, in some embodiments, the amount of Wear Particles in the formulation should be between about 1% and 10% of the amount of wear particles generated over a one month's span by the prosthesis with which the formulation will be used.

In some embodiments, the formulation consists essentially of the Th2-inducing adjuvant. In these embodiments, the formulation is placed adjacent an articulation surface of the prosthesis, and Wear Particles produced from the articulation interface flow into the formulation consisting essentially of the Th2-inducing adjuvant, thereby producing an adjuvant/wear particle mixture. Local collagen proteins then adhere to the wear particles and present novel epitopes to local antigen presenting cells responding to this mixture.

The Th2-inducing adjuvant of the present invention may be provided in many forms. In some embodiments, it is delivered systemically or injected intramuscularly as a vaccine. In some embodiments, it is delivered locally to a region containing the articulating prosthetic.

In some embodiments, the adjuvant is delivered as a powder. In some embodiments, it is delivered as a fluid, and preferably as a gel.

Although alum is the preferred adjuvant for inducing a Th2 immune response, it is believed that other metallic hydroxides may also be effective in inducing the desired Th2 response. Preferably, the metallic cation of the metallic hydroxide is divalent or trivalent.

In some of these embodiments, the formulation comprises a gel comprising alum and UHMWPE Wear Particles.

In some embodiments, the Th2-inducing adjuvant is present in the formulation in a concentration of between about 1 μg/ml and 5 mg/ml. Whereas the higher concentrations in this range are similar to those typically used in a systemic vaccine, the lower end of this concentration range is used in in vitro experiments in order to avoid cytotoxicity. Accordingly, if the formulation is used as a systemic vaccine, then the higher end of the concentration range may be selected. Conversely, if the formulation is provided locally (e.g., as a coating upon the prosthetic), then the lower end of the conventration range may be selected. Preferably, when the formulation is provided locally, the Th2-inducing adjuvant is present in the formulation in a concentration of between about 1 μg/ml and 10 μg/ml.

The formulation of the present invention may be delivered at any number of times. For example, it may be delivered to the patient prior to surgery. In these embodiments, it may be delivered systemically or locally. The formulation may be delivered at the time of implant surgery. In these embodiments, the formulation be provided as either an injection or as a coating upon a portion of the prosthetic. In some embodiments, it may be delivered after surgery. In these embodiments, it is preferably delivered as an injection into an osteolytic region.

In some embodiments, collagen is added to the formulation of the present invention. Collagen typically has a hydrophobic tail. It is believed that this tail of the added collagen will complex with the Wear Particles in the same manner that the patient's collagen complexes with wear particles produced from a prosthesis, and thereby change the presentation of the collagen to the immune system to provide novel epitopes to the immune system.

In some embodiments, the collagen is provided in a soluble form. In some embodiments, the collagen is provided in a fibrillar form. The fibrillar form is preferred because it can be used in a slurry and thereby help keep the wear particles localized.

In some embodiments, the collagen is recombinant human collagen. Providing human collagen will minimize the variances between the added collagen and the collagen of the patient, and so will allow the formulation to mimic as closely as possible the natural complexation that typically occurs during osteolysis.

Preferably, the collagen is selected from the group consisting of types I, type II, type IV and type V collagen. Type II collagen is particularly preferred.

In some embodiments, antigen presenting cells are added to the formulation. In some embodiments, thereof concentrated, immature dendritic cells may be added to the formulation in order to enhance the antigen presenting function. Preferably, the dendritic cells are provided by the patient's blood or bone marrow, and may be concentrated by conventional methods, including the centrifugal elutriation procedure disclosed in Ossevoort, J. Immunological Methods, 155, 1992, 101-111.

In other embodiments, concentrated macrophages may be added (from a buffy coat) in order to enhance the antigen presenting function. Preferably, the macrophages are provided by the patient's blood or bone marrow, and may be concentrated by conventional methods, including the centrifugation.

TGF-β can be added to help convert the immature dendritic cells to DC2 cells and to drive the polarization of the immune response to Th2. Liu, Nature Immunology, 2(7), July 2001 585-589, and King, Immunity, 8, May 1998, 601-613. Therefore, in some embodiments, the formulation additionally comprises an effective amount of TGF-β. In some embodiments thereof, the TGF-β is obtained from platelets from the patient's blood. In other embodiments thereof, the TGF-β is exogenous.

It is known that both IL-4 and Il-10 can help determine a Th2 immune response. Therefore, in some embodiments, the formulation additionally comprises an effective amount of an interleukin capable of inducing a Th2 response, and is preferably selected from the group consisting of IL-4 and IL-10.

In addition, if the formulation is provided substantially after the time of implant surgery, so that the patient has already experienced substantial osteolysis, then it may be helpful to add therapeutic proteins, such as IL-10 or TGF-β, to the formulation as well.

In some embodiments, the active immunization of this invention is directed against osteolysis occurring due to wear debris (preferably UHMWPE wear debris) from a hip joint prosthesis (preferably an acetabular cup). In some embodiments thereof, the acetabular cup is selected from the cups disclosed in U.S. Pat. Nos. 5,282,864; 6,017975; and 6,228,900, the specifications of which are incorporated by reference in their entireties.

In some of these hip joint embodiments, the formulation may be placed upon a non-articulating surface of the acetabular cup. In some embodiments, the non-articulating surface is the rim of the acetabular cup surrounding the articulation surface. In some embodiments, the non-articulating surface is the outer surface of the acetabular cup. In some embodiments, the acetabular cup is modular and comprises an inner liner (preferably, UHMWPE) and an outer backing (preferably, a metal backing), and the formulation is placed at the interface of the inner liner and the outer backing. In some embodiments, the modular acetabular cup comprises a cavity (preferably opening out upon the backside surface of the cup) and the formulation is placed in the cavity. In some embodiments, the cavity is a through-hole traversing the rim and backside of the cup In some embodiments, the active immunization of this invention is directed against osteolysis occurring due to wear debris (preferably UHMWPE wear debris) from a knee joint prosthesis (preferably a tibial insert upon a tibial tray).

In some of these knee joint embodiments, the formulation may be placed upon a non-articulating surface of the tibial component. In some embodiments, the tibial component is modular and comprises an articulation insert (preferably, UHMWPE) and an outer tibial tray (preferably, a metal backing). In some embodiments, the non-articulating surface upon which the formulation is placed is the rim of the tibial tray. In some embodiments, the non-articulating surface upon which the formulation is placed is the outer surface of the tibial tray. Preferably, the formulation is placed at the interface of the articulation insert and the tibial tray. In some embodiments, the modular tibial component comprises a cavity (preferably opening out upon the backside surface of the tibial tray) and the formulation is placed in the cavity.

In some embodiments, the active immunization of this invention is directed against lysis occurring due to wear debris (preferably UHMWPE wear debris) from an intervertebral motion disc prosthesis (preferably a cervical motion disc).

In some of these cervical disc prosthesis embodiments, the formulation may be placed upon a non-articulating surface of an articulating component. In some embodiments, the one of the articulating components is modular and comprises an articulation insert (preferably, UHMWPE) and an outer tray (preferably, a metal backing). In some embodiments, the non-articulating surface upon which the formulation is placed is the rim of the outer tray. In some embodiments, the non-articulating surface upon which the formulation is placed is the outer surface of the outer tray. Preferably, the formulation is placed at the interface of the articulation insert and the outer tray. In some embodiments, the modular articulation component comprises a cavity (preferably opening out upon the backside surface of the component) and the formulation is placed in the cavity.

In some embodiments, the active immunization of this invention is directed against lysis occurring due to wear debris from fixation components, such as pedicle screws or intramedullary rods, that have loosened over time.

In some embodiments, the active immunization of this invention is directed against lysis occurring due to wear debris from a small joint prosthetic component, such as a finger joint prosthetic.

In some embodiments, the active immunization of this invention is directed against lysis occurring in a patient having osteoporosis. These patients are considered high risk patients.

In some embodiments, the active immunization of this invention is directed against lysis occurring in a patient having an infected implant. These patients are considered high risk patients.

In some embodiments, there is provided a kit comprising:

a) a formulation adapted to elicit a Th2 immune response, and

b) an orthopedic implant having an articulation interface.

This kit is generally used at the time of surgery, wherein the implant is implanted and the formulation is administered in the vicinity of the implant.

In some embodiments thereof, the orthopedic implant is a hip joint prosthesis, preferably an acetabular cup having a UHMWPE liner. In others, the orthopedic implant is a knee joint prosthesis, preferably a tibial component having a UHMWPE insert. Preferably, the formulation comprises: a) a Th2-inducing adjuvant, and b)Wear Particles, wherein the adjuvant comprises a metallic cation, preferably alum in the form of a gel. Preferably, the Wear Particles comprise a polymer, such as polyethylene, titanium, or cobalt-chrome.

Sciatica is largely produced when nucleus pulposus is exuded from the immune privileged environment of the intervertebral disc and contacts a nerve root. The immune system recognizes the exuded material as foreign and produces a Th1-type inflammatory response in the area of the nerve root which includes the introduction of activated macrophages. It is believed that Th1 cytokines such as TNF-a are emitted by these macrophages and combine with receptors on the nerve root to produce pain.

The present inventors believe that polarization of the sciatica-related inflammatory response to a Th2 response would condition the patient to the exuded nucleus pulposus and thereby alleviate sciatica.

Accordingly, in accordance with the present invention, there is provided a method of preventing or treating sciatica from a nucleus pulposus of a patient, comprising the steps of:

a) administering to the patient a formulation comprising:

i) a Th2-inducing adjuvant, and

ii) nucleus pulposus.

What is claimed is:

1. A method of preventing or treating osteolysis comprising the steps of:
a) administering to a patient a formulation comprising:
i) a Th2-inducing adjuvant, and
ii) Wear Particles.

2. The method of claim 1 wherein the formulation is injected into the patient.

3. The method of claim 1 wherein the formulation is implanted into the patient through an incision.

4. The method of claim 3 wherein an orthopedic implant capable of producing Wear Particles is implanted into the patient through the incision.

5. The method of claim 4 wherein the formulation is attached to the implant during implantation of the implant.

6. The method of claim 1 wherein an orthopedic implant capable of producing Wear Particles is implanted into the patient prior to the administration.

7. The method of claim 1 wherein an orthopedic implant capable of producing Wear Particles is implanted into the patient after the administration.

8. The method of claim 1 wherein the adjuvant comprises a metallic hydroxide component.

9. The method of claim 8 wherein the adjuvant is alum.

10. The method of claim 1 wherein the formulation further comprises iii) collagen.

11. A formulation for treating or preventing osteolysis caused by Wear Particles, comprising:
i) a Th2-inducing adjuvant, and
ii) Wear Particles.

12. The formulation of claim 11 wherein the adjuvant comprises a metallic hydroxide.

13. The formulation of claim 11 wherein the formulation further comprises iii) collagen.

14. The formulation of claim 11 provided in the form of a gel.

15. The formulation of claim 11 wherein the Wear Particles have a D₅₀ particle size of less than 10 microns.

16. The formulation of claim 11 wherein the Wear Particles comprises a polymer.

17. The formulation of claim 16 wherein the polymer is polyethylene.

18. The formulation of claim 11 wherein the Wear Particles comprise titanium.

19. The formulation of claim 11 wherein the Wear Particles comprise cobalt-chrome.

20. The formulation of claim 11 wherein the Wear Particles comprises PMMA.

21. A kit comprising:
a) a formulation adapted to elicit a Th2 immune response, and
b) an orthopedic implant having an articulation interface.

22. The kit of claim 21 wherein the orthopedic implant is a hip joint prosthesis.

23. The kit of claim 22 wherein the orthopedic implant is an acetabular cup.

24. The kit of claim 23 wherein the acetabular cup is a UHMWPE liner.

25. The kit of claim 21 wherein the orthopedic implant is a knee joint prosthesis.

26. The kit of claim 22 wherein the knee joint prothesis is a tibial component.

27. The kit of claim 23 wherein the tibial component is a UHMWPE insert.

28. The kit of claim 21 wherein the formulation comprises:
i) a Th2-inducing adjuvant, and
ii) Wear Particles.

29. The kit of claim 28 wherein the adjuvant comprises a metallic hydroxide.

30. The kit of claim 28 wherein the adjuvant comprises alum.

31. The kit of claim 28 wherein the formulation is provided in the form of a gel.

32. The kit of claim 28 wherein the Wear Particles comprises a polymer.

33. The kit of claim 28 wherein the polymer is polyethylene.

34. The kit of claim 28 wherein the Wear Particles comprise titanium.

35. The kit of claim 28 wherein the Wear Particles comprise cobalt-chrome.

36. An orthopedic implant having surface having a coating thereon, wherein the coating is adapted to elicit a Th2 immune response.

37. The implant of claim 36 wherein the coating comprises a metallic cation.

38. The implant of claim 37 wherein the coating comprises alum.

39. The implant of claim 36 wherein the coating is provided in the form of a gel.

40. The implant of claim 36 wherein the surface having the coating is an outer surface.

41. The implant of claim 36 wherein the surface having the coating is an inner surface.

42. A formulation for treating or preventing osteolysis caused by Wear Particles, comprising:
a) a Th2-inducing adjuvant, and
b) particles selected from the group consisting of a polymer, titanium, titanium alloy, cobalt chrome, and PMMA, and mixtures thereof.

43. The formulation of claim 42 further comprising:
c) collagen.

44. The formulation of claim 43 wherein the collagen comprises type II collagen.

45. The formulation of claim 43 wherein the collagen comprises fibrillar collagen.

46. The formulation of claim 43 wherein the formulation further comprises d) a therapeutic protein.

47. The formulation of claim 42 wherein the particles comprise a mixture of polymer and titanium alloy particles.

48. A formulation for treating or preventing osteolysis caused by Wear Particles, comprising:
a) a Th2-inducing adjuvant, and
b) particles having a D50 particle size of between 0.1 um and 10 um.

49. A method of preventing or treating sciatica from a nucleus pulposus of a patient, comprising the steps of:
a) administering to the patient a formulation comprising:
i) a Th2-inducing adjuvant, and
ii) nucleus pulposus.
